(19) United States
(12) Patent Application Publication
(10) Pub. No.: US 2023/0032701 A1
(43) Pub. Date: Feb. 2, 2023

(54) IMPLANT, IMPLANT COMPONENT AND METHOD FOR THE PRODUCTION THEREOF

(71) Applicant: FRAUNHOFER-GESELLSCHAFT ZUR FÖRDERUNG DER ANGEWANDTEN FORSCHUNG E.V., München (DE)

(72) Inventor: Harald HOLECZEK, Hohentengen am Hochrhein (DE)

(73) Assignee: FRAUNHOFER-GESELLSCHAFT ZUR FÖRDERUNG DER ANGEWANDTEN FORSCHUNG E.V., München (DE)

(21) Appl. No.: 17/784,178

(22) PCT Filed: Dec. 9, 2020
(86) PCT No.: PCT/EP2020/085181

§ 371 Date: Jun. 10, 2022

(30) Foreign Application Priority Data
Dec. 11, 2019 (DE) 10 2019 219 355.2

(51) Int. Cl. A61F 2/28; A61L 27/54; A61L 27/30; A61L 27/32

(52) U.S. Cl. CPC A61F 2/28; A61L 27/54; A61L 27/306; A61L 27/32; A61F 2002/2821; A61L 2300/102; A61L 2300/404; A61L 2420/02; A61L 2420/06

(57) ABSTRACT

An implant and/or an implant component is made available, having a main body which, at least on a surface, contains or consists of an electrically conductive material, and having a layer of calcium hydroxide applied to the electrically conductive material of the main body. The implant or the implant component is characterized in that the layer of calcium hydroxide contains calcium phosphate, specifically in a percentage by weight that is less than the percentage by weight of calcium hydroxide in this layer. A method for making available the implant according to the invention or the implant component according to the invention is also proposed. The implant made available and the implant component made available are characterized in that they have a local and temporary antimicrobial action, prevent formation of antibiotic-resistant microorganisms, act on bone substance in a manner that promotes growth, and produce no adverse side effects in the body.

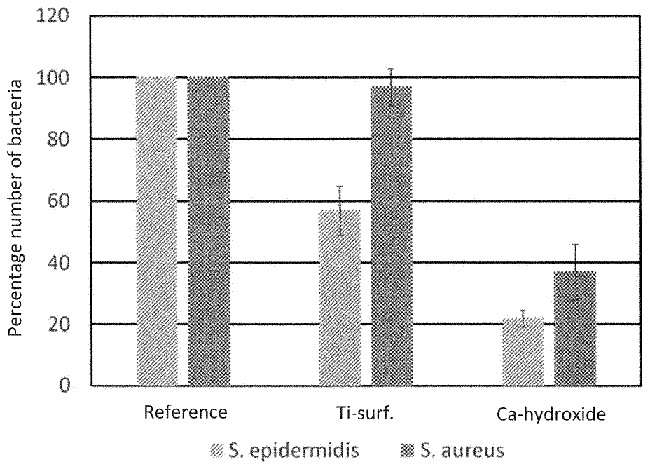

An implant and/or an implant component is provided that has a base body comprising or consisting of an electrically conductive material on at least a surface, and that has a calcium hydroxide layer deposited on the electrically conductive material of the base body. The implant or implant component is characterized in that the calcium hydroxide layer comprises calcium phosphate in a weight percentage which is less than the weight percentage of calcium hydroxide in said layer. A method for providing the implant according to the invention or the implant component according to the invention is further presented. The provided implant or the provided implant component are characterized in that they have a local and temporary antimicrobial effect, prevent a formation of antibiotic-resistant germs, have a growth-promoting effect on bone material and do not cause any negative side effects in the body.

Implant infections are a serious problem in surgical care, including hip and knee implants. According to the Australian National Register of Joint Implants, the rate of infection following implant placement is between one and three percent of all surgeries. In primary surgeries, infection rates of two and a half percent have been observed and in revision surgeries, even infection rates of up to ten percent. Since patients with joint implants are getting younger and younger, the number of revision surgeries and thus the risk of infection after implant placement will increase in the future.

Implant infections are serious complications having an increased risk of mortality and significant socio-economic costs. They require the removal of the implant from the body and its replacement with a new implant, that is, a repeat surgical intervention. In connection with the surgical intervention, various disease consequences can occur when (re)inserting an implant. Infections are possible, because surgeries are not completely sterile and infection is often caused by touching the edges of the wound, hematogenously or aerogenously (see Gravius, S., Wirtz, D. C., Orthopädie, 2015, vol. 44, pp. 952-960), the edges not being completely free of germs due to the preparation for the surgery. The infection can often occur with only a low number of germs and is controlled in most cases by the patient's immune system and antibiotic prophylaxis. However, this is often not the case, especially in high-risk patients with comorbidities, and the risk of infection is significantly increased in these patients.

Various approaches are taken in the prior art in order to reduce the risk of infection in implants.

It is known to coat implants with antibiotics (Schmidmaier, G. et al., Injury, 2006, Volume 37, Suppl. 2, p. 105-p. 112) or to incorporate antibiotics into a coating on the implant (Hetrick, E. M. et al., Chem. Soc. Rev., 2006, Vol. 35, Issue 9, pp. 780-789), the antibiotics being slowly released from the coating over time. For implants which are fixed in the bone with the aid of cement, the cement can also be enriched with antibiotics (Parvizi, J. et al, Acta Orthopaedica, 2008, Vol. 79, p. 335-341). The use of antibiotics is sometimes problematic, since germs located on the implant can already be resistant to the antibiotics used, making the antibiotic ineffective. In addition, the antibiotics released from the layer over a longer period of time remain in the body for a long time and can thus also contribute to the development of resistance in bacterial strains. The development of resistance can also affect bacterial strains that are of no importance for the ingrowth of the implant.

Impregnation of the implant surface with silver particles, partly as silver nanoparticles, has been used for a few years to prevent infections (Knetch, M. L. W. et al., Polymers, 2011, Vol. 3, pp. 340-366). However, the use of silver is problematic, since it is still not fully understood which, perhaps unwanted, effects the silver particles have on the body and what long-term effects said particles have.

For these reasons, there is a strong motivation to implement an antibacterial finish on implant surfaces or implant component surfaces in a way that has reliable, high antimicrobial effectiveness but does not cause any harmful side effects in the body. Ideally, the implants or the implant components should also have a growth-promoting effect on bone material, since this accelerates the healing process.

Starting therefrom, it was the object of the present invention to provide an implant or an implant component that does not have the disadvantages known in the prior art. In particular, the implant or the implant component should only have a local and only temporary antimicrobial effect, avoid the formation of antibiotic-resistant germs, exert a growth-promoting effect on bone material and not cause any side effects in the body.

The object is achieved by the implant and/or the implant component having the features of claim 1 and by the method for manufacturing an implant and/or an implant component having the features of claim 9. The dependent claims show advantageous developments.

According to the invention, an implant or an implant component is provided, comprising

a) a base body of an implant or an implant component, comprising or consisting of an electrically conductive material at least on a surface; and

b) a calcium hydroxide layer comprising or consisting of calcium hydroxide (Ca(OH)₂), the calcium hydroxide layer being deposited on the electrically conductive material of the base body;

characterized in that the calcium hydroxide layer comprises calcium phosphate in a weight percentage less than the weight percentage of calcium hydroxide, the weight percentage being based on the total weight of the calcium hydroxide layer.

The calcium hydroxide layer of the implant or the implant component causes an antimicrobial (for example, bactericidal) effect without side effects in the body to be achieved only locally and only for a limited time by increasing the pH value, preventing the formation of antibiotic-resistant germs and providing a growth-promoting effect for bone material (positive osseointegrative properties). The reason for the only temporary and local effect is that the active ingredients in the calcium hydroxide layer are completely dissolved and transformed by the body fluids in situ after a certain period of time.

However, the effect of the calcium hydroxide layer is long and strong enough to ensure an antimicrobial effect for a sufficiently long time after the implant or the implant component has been inserted into the body and to kill any germs that enter the body, for example, during the insertion of the implant or the implant component (during surgery). The only temporary effect of the calcium hydroxide layer avoids long-term risks to the health of the patient. Even if not all germs are killed by the calcium hydroxide layer, the number of germs is reduced so much by the effect of the calcium hydroxide layer that the body's own immune system can easily take over the fight against the remaining germs.

The calcium phosphate present in the calcium hydroxide layer in addition to the calcium hydroxide also has a bactericidal effect, supports the effect of increasing the pH value by the calcium hydroxide and has a growth-promoting effect on bone material in the vicinity of the implant or the implant component. The calcium phosphate thus supports the osseointegration of the implant or the implant component and thus causes faster and more reliable integration in the body after it has been placed in the body.

The implant according to the invention or the implant component according to the invention achieve the antimicrobial effect without the use of antibiotics or metals having an antibacterial effect, such as silver, copper or strontium. This prevents the formation of antibiotic-resistant germs after said implant or implant component has been placed in the body. In addition, the antibacterial calcium hydroxide layer can be completely resorbed by the body, so that, unlike antibiotic coatings or metal coatings, there are no long-term risks to the patient's health.

The calcium phosphate comprised in the calcium hydroxide layer can comprise or consist of a calcium phosphate selected from the group consisting of calcium dihydrogen phosphate (“monocalcium phosphate”=Ca(H₂PO₄)₂), calcium hydrogen phosphate (“dicalcium phosphate”=CaHPO₄), calcium phosphate (“tricalcium phosphate”=Ca₃(PO₄)₂), hydroxyapatite (Ca5[OH|(PO₄)₃]), brushite (CaH—PO₄·2H₂O) and combinations thereof. The calcium phosphate of the calcium hydroxide layer preferably comprises or consists of hydroxyapatite and/or brushite.

The implant or the implant component can be characterized in that the base body comprises or consists of a material selected from the group consisting of metals, semi-metals, carbons, plastics and ceramics. The material is preferably selected from the group of metals. In particular, the material is titanium, a titanium alloy or cobalt-chromium steel.

The electrically conductive material of the base body can be deposited on the base body or be monolithic (in one piece) with the base body. Said electrically conductive material can comprise or consist of a material that is different from the base body or comprise or consist of a material that is identical to the base body.

According to the invention, the “surface” of the base body comprising or consisting of the electrically conductive material is understood to mean a surface of the base body to which either the electrically conductive material (via a specific method) is deposited (two-piece structure between the base body and the electrically conductive material) or in which the electrically conductive material is (already) an integral part (base body and the electrically conductive material being one single piece).

For the purposes of this invention, electrically conductive material is also understood to mean materials which are electrically conductive due to their chemical nature and, when they come into contact with oxygen (for example, via contact with air or water), form an oxidative protective layer on their surface that is not electrically conductive. This includes, for example, the materials titanium and/or titanium alloys.

The electrically conductive material of the base body can be selected from the group consisting of metals, semi-metals, carbons and plastics. The electrically conductive material is preferably selected from the group of metals, particularly preferably selected from the group consisting of titanium, titanium alloy, tantalum, magnesium and alloys thereof. In particular, the electrically conductive material is titanium or a titanium alloy.

A calcium phosphate layer comprising or consisting of calcium phosphate can be deposited on the electrically conductive material of the base body.

The calcium phosphate of the calcium phosphate layer can be a calcium phosphate selected from the group consisting of calcium dihydrogen phosphate (“monocalcium phosphate”=Ca(H₂PO₄)₂), calcium hydrogen phosphate (“dicalcium phosphate”=CaHPO₄), calcium phosphate (“tricalcium phosphate”=Ca3(PO₄)₂), hydroxyapatite (Ca₅[OH|(PO₄)₃]), brushite (CaHPO₄·2H₂O) and combinations thereof. The calcium phosphate of the calcium hydroxide layer preferably comprises or consists of hydroxyapatite and/or brushite. Such a calcium phosphate layer can be provided, for example, as described in Dorozhkin, S. V. et al. (Progress in biomaterials, Vol. 1, Issue 1, p. 1ff.). The calcium phosphate layer has the advantage that the improvement in ingrowth behavior (that is, the positive osseointegrative properties), which is already provided by the proportion of calcium phosphate in the calcium hydroxide layer, is improved even further.

The calcium phosphate layer preferably comprises calcium phosphate in a weight percentage which is higher than a weight percentage of calcium hydroxide in said layer, the weight percentage being based on the total weight of the calcium phosphate layer.

In a preferred embodiment, the calcium phosphate layer comprises >50% by weight calcium phosphate, based on the total weight of the calcium phosphate layer.

In a preferred embodiment, the calcium phosphate layer makes contact with the electrically conductive material of the base body. The calcium phosphate layer here can cover the electrically conductive material of the base body (for example, a sprayed titanium layer). In this case, the roughness of the electrically conductive material is slightly reduced. However, the morphology of the electrically conductive layer changes only slightly here. In addition, the surface is still self-similar in its geometric structure, that is, strongly structured, broken up in many ways and comprising recurring geometric elements on different scales.

In a further preferred embodiment, the calcium phosphate layer makes contact with the calcium hydroxide layer.

The calcium phosphate layer can be deposited on the electrically conductive material of the base body by means of an electrochemical deposition or a plasma spraying process.

The calcium phosphate layer is preferably present as a layer which has the same layer thickness over the entire extent of the layer.

In addition, the calcium phosphate layer can be present as a porous layer.

In a preferred embodiment, the calcium phosphate layer has a layer thickness in the range from 2 to 500 μm, preferably in the range from 5 to 200 μm, particularly preferably in the range from 10 to 130 μm.

The calcium hydroxide layer of the implant or the implant component can be deposited by electrochemical deposition, preferably by electrochemical deposition with an electrolyte that comprises calcium nitrate, ammonium dihydrogen phosphate and citric acid and particularly preferably has a pH value of <7. Compared to other deposition methods, the electrochemically deposited calcium hydroxide layer has the advantage that the layer is also deposited in any pores and cavities of the electrically conductive material of the base body and thus has good adhesion. Furthermore, an electrochemically deposited layer has a high level of homogeneity, a fine layer structure and a constant layer thickness over the entire extent of the layer. Homogeneous effectiveness can consequently be ensured in a reliable manner by the electrochemical deposition. In addition, the uppermost region of the layer deposited via electrochemical deposition can be very easily dissolved by body fluids after the implant or implant component has been placed in the body. Consequently, the effect of increasing the pH value, and thus the bactericidal effect, can set in very quickly after being placed in the body.

The calcium hydroxide layer is preferably present as a layer which has the same layer thickness over the entire extent of the layer.

In a preferred embodiment, the calcium hydroxide layer has a layer thickness in the range from 1 to 50 μm, preferably in the range from 2 to 40 μm, particularly preferably in the range from >20 to 30 μm (for example, in the range from 22 to 30 μm). The greater the layer thickness of the calcium hydroxide, the stronger and longer lasting is the antimicrobial effect of said layer. This is because, in a given volume of fluid in the body, a thicker layer can create a higher pH value and remain stable longer, thus allowing a stronger and longer-lasting antimicrobial effect to develop in situ.

In a preferred embodiment, the proportion of calcium hydroxide in the calcium hydroxide layer is in the range of >50% by weight and <100% by weight, based on the total weight of the calcium hydroxide layer. Consequently, the proportion of calcium phosphate is preferably in the range from >0% by weight to <50% by weight, based on the total weight of the calcium hydroxide layer. In the calcium hydroxide layer, the proportion of calcium phosphate is particularly preferably in the range from >0% by weight and <40% by weight, preferably from >2% by weight and <20% by weight, based on the total weight of the calcium hydroxide layer.

The calcium phosphate is particularly preferably present in the calcium hydroxide layer in the form of particles, in particular in the form of particles having a particle size of <1 μm. The particle size can be determined, for example, via electron microscopy.

In a preferred embodiment, the implant according to the invention or the implant component according to the invention is intended for use in medicine, preferably for use in surgery.

According to the invention, a method for manufacturing an implant or an implant component is also presented. The method comprises the steps

a) providing a base body of an implant or an implant component, the base body comprising or consisting of an electrically conductive material at least on a surface; and

b) depositing a calcium hydroxide layer comprising or consisting of calcium hydroxide (Ca(OH)₂) to the electrically conductive material of the base body;

characterized in that the calcium hydroxide layer is deposited so that it comprises calcium phosphate in a weight percentage which is less than the weight percentage of calcium hydroxide, the weight percentage being based on the total weight of the calcium hydroxide layer.

Using the method, a calcium hydroxide layer is produced, the calcium hydroxide layer partly comprising or consisting of calcium hydroxide on the one hand and partly calcium phosphate on the other hand. The calcium phosphate comprised in the layer simultaneously supports osseointegration.

The calcium phosphate comprised in the calcium hydroxide layer can be calcium phosphate selected from the group consisting of calcium dihydrogen phosphate (“monocalcium phosphate”=Ca(H₂PO₄)₂), calcium hydrogen phosphate (“dicalcium phosphate”=CaHPO₄), calcium phosphate (“tricalcium phosphate”=Ca₃(PO₄)₂), hydroxyapatite (Ca₅[OH|(PO₄)₃]), brushite (CaHPO₄·2H₂O) and combinations thereof. The calcium phosphate of the calcium hydroxide layer preferably comprises or consists of hydroxyapatite and/or brushite.

In the method, a base body can be provided that comprises or consists of a material selected from the group consisting of metals, semi-metals, carbons, plastics and ceramics. The material is preferably selected from the group of metals, particularly preferably selected from the group of light metals. In particular, the material is titanium or a titanium alloy.

Furthermore, a base body can be provided in the method, the electrically conductive material of which is deposited on the base body or is monolithic (in one piece) with the base body. The electrically conductive material can comprise or consist of a material that is different from the base body or comprise or consist of a material that is identical to the base body.

The electrically conductive material can be selected from the group consisting of metals, semi-metals, carbons and plastics. The electrically conductive material is preferably selected from the group of metals, particularly preferably from the group consisting of titanium, titanium alloy, tantalum, magnesium and alloys thereof. In particular, the electrically conductive material is titanium or a titanium alloy.

In a preferred embodiment of the method, a calcium phosphate layer comprising or consisting of calcium phosphate is deposited on the electrically conductive material of the base body.

The calcium phosphate of the calcium hydroxide layer can be calcium phosphate selected from the group consisting of calcium dihydrogen phosphate (“monocalcium phosphate”=Ca(H₂PO₄)₂), calcium hydrogen phosphate (“dicalcium phosphate”=CaHPO₄), calcium phosphate (“tricalcium phosphate”=Ca₃(PO₄)₂), hydroxyapatite (Ca₅[OH|(PO₄)₃]), brushite (CaHPO₄·2H₂O) and combinations thereof. The calcium phosphate of the calcium hydroxide layer preferably comprises or consists of hydroxyapatite and/or brushite. Such a calcium phosphate layer can be produced, for example, by a method as described in Dorozhkin, S. V. et al. (Progress in biomaterials, Vol. 1, Issue 1, p. 1ff.). The calcium phosphate layer has the advantage that the improvement in ingrowth behavior (that is, the positive osseointegrative properties), which is already provided by the proportion of calcium phosphate in the calcium hydroxide layer, is improved even further.

The calcium phosphate layer preferably comprises calcium phosphate in a weight percentage which is higher than a weight percentage of calcium hydroxide in said layer, the weight percentage being based on the total weight of the calcium phosphate layer.

In a preferred embodiment, the calcium phosphate layer comprises >50% by weight calcium phosphate, based on the total weight of the calcium phosphate layer.

In a preferred embodiment, the calcium phosphate layer is deposited on the base body such that said calcium phosphate layer contacts the electrically conductive material of the base body. The calcium phosphate layer here can cover the electrically conductive material of the base body (for example, a sprayed titanium layer). This slightly reduces the roughness of the electrically conductive material. However, the morphology of the electrically conductive layer is changed only slightly. In addition, the surface is still self-similar in its geometric structure, that is, strongly structured, broken up in many ways and comprising recurring geometric elements on different scales.

In a further preferred embodiment, the calcium phosphate layer is deposited on the base body such that said calcium phosphate layer makes contact with the calcium hydroxide layer.

The calcium phosphate layer can be deposited on the electrically conductive material of the base body by means of an electrochemical deposition or a plasma spraying process.

The calcium phosphate layer is preferably deposited as a layer that has the same layer thickness over the entire extent of the layer.

In addition, the calcium phosphate layer can be deposited as a porous layer.

In a preferred embodiment, the calcium phosphate layer is deposited up to a layer thickness in the range from 2 to 500 μm, preferably in the range from 5 to 200 μm, particularly preferably in the range from 10 to 130 μm.

The calcium hydroxide layer deposited in the process can be deposited by electrochemical deposition, preferably via electrochemical deposition with an electrolyte comprising calcium nitrate, ammonium dihydrogen phosphate and citric acid and particularly preferably having a pH value of <7. This method has the advantage that the layer is also deposited in any pores and cavities of the electrically conductive material of the base body and thus has good adhesion. Furthermore, the electrochemically deposited layer has a high level of homogeneity, a fine layer structure and a constant layer thickness over the entire extent of the layer. Homogeneous effectiveness can consequently be ensured in a reliable manner by the electrochemical deposition. In addition, the uppermost region of the layer deposited via electrochemical deposition can be very easily dissolved by body fluids after the implant or implant component has been placed in the body. The effect of increasing the pH value, and thus the bactericidal effect, can thus set in very quickly after being placed in the body.

In the method, the calcium hydroxide layer is preferably deposited as a layer having the same layer thickness over the entire extent of the layer.

In a preferred embodiment, the calcium hydroxide layer is deposited up to a layer thickness in the range from 1 to 50 μm, preferably in the range from 2 to 40 μm, particularly preferably in the range from >20 to 30 μm (for example, 22 to 30 μm).

In a preferred embodiment, the proportion of calcium hydroxide in the calcium hydroxide layer is in the range of >50% by weight and <100% by weight, based on the total weight of the calcium hydroxide layer. Consequently, the proportion of calcium phosphate is preferably in the range from >0% by weight to <50% by weight, based on the total weight of the calcium hydroxide layer. The calcium hydroxide layer is particularly preferably deposited such that the proportion of calcium phosphate in the calcium hydroxide layer is in the range from >0% by weight and <40% by weight, preferably from >2% by weight and <20% by weight, based on the total weight of the calcium hydroxide layer.

The calcium hydroxide layer is particularly preferably deposited such that the calcium phosphate is present in the form of particles in the calcium hydroxide layer.

In a preferred embodiment, the calcium hydroxide layer is deposited such that the calcium phosphate is present in the form of particles having a particle size in the range of <1 μm.

It is particularly preferred that an implant according to the invention or an implant component according to the invention is manufactured using the method presented here.

The subject according to the invention is to be explained in more detail on the basis of the following FIGURE and the following examples, without wishing to restrict it to the specific embodiments shown here.

The FIGURE shows the result of an experiment in which the degree of reduction in the number of bacteria of two bacterial strains (S. epidermis and S. aureus) was measured as a function of the surface of an implant to which the bacterial strains were respectively deposited. The reference is an implant having a polyethylene surface (without antibacterial properties). It has been found that an implant surface made of titanium leads to a certain reduction in the number of bacteria only in S. epidermis. However, a surface comprising calcium hydroxide led to a significant reduction in the number of bacteria in both bacterial strains.

### Example 1—Manufacture of an Implant According to the Invention without a Calcium Phosphate Intermediate Layer

An implant is used, the base body of which consists of a titanium alloy. The base body is coated with two layers of pure titanium in a vacuum. The first layer (adhesive layer) has a thickness of 30 μm and the second layer has a thickness of 150 μm. Said sprayed titanium layer forms an electrically conductive layer and is then provided with a calcium hydroxide layer in an aqueous electrolyte.

The composition of the aqueous electrolyte and the process parameters used are listed in Table 1.

TABLE 1

| Electrolyte | Ca(NO₃)₂ | 75 | mmol/l |
|---|---|---|---|
| | (NH₄)H₂PO₄ | 40 | mmol/l |
| | Citric acid | 20 | mmol/l |
| Process temperature | | 60° C. | |
| Current density | | 90 | mA/cm² |
| Time | | 20 | min. |
| Layer thickness | | 15 | μm |

The deposited calcium hydroxide layer comprises not only calcium hydroxide but also calcium phosphate. The reason for this is that calcium hydroxide constitutes an intermediate phase in the electrochemical deposition of calcium phosphate. The presence of citric acid in the electrolyte suppresses the formation of calcium phosphate, but in the specified concentration only to such an extent that a calcium hydroxide layer still comprising calcium phosphate forms. However, the proportion of calcium phosphate is lower than the proportion of calcium hydroxide.

The ratio of the two material components can be shifted by changing the amount of citric acid used. This means that with a higher concentration of citric acid (or citrate ions) in the electrolyte, the amount of deposited calcium phosphate decreases and with a lower concentration of citric acid (or citrate ions) the amount of deposited calcium phosphate increases. A higher proportion of calcium phosphate reduces the bactericidal effect a little, but the osseointegrative effect of the calcium phosphate increases, which is desired according to the invention. Very high concentrations of citrate ions in the electrolyte are also disadvantageous since said concentrations produce a lower pH value in the aqueous electrolyte and thus cause the deposited layer to redissolve more quickly, whereby the maximum achievable layer thickness decreases before a dynamic equilibrium is established.

The electrolyte described in Table 1 has a concentration of citrate ions that is advantageous since, on the one hand, it provides a high content of calcium hydroxide in the layer and, on the other hand, it ensures that only a small redissolution of the deposited layer is effected and that the deposited layer also comprises calcium phosphate.

### Example 2—Manufacture of an Implant According to the Invention with a Calcium Phosphate Intermediate Layer

An implant is used, the base body of which consists of a titanium alloy. The base body is coated with two layers of pure titanium in a vacuum. The first layer has a thickness of 30 μm and the second layer has a thickness of 50 μm. Said sprayed titanium layer forms an electrically conductive layer and is then provided with a calcium phosphate layer having a thickness of 100 μm in a vacuum.

The implant equipped with the calcium phosphate layer is then provided with a calcium hydroxide layer in an aqueous electrolyte.

The composition of the aqueous electrolyte and the process parameters used are listed in Table 2.

TABLE 2

| Electrolyte | Ca(NO₃)₂ | 75 | mmol/l |
|---|---|---|---|
| | (NH₄)H₂PO₄ | 40 | mmol/l |
| | Citric acid | 20 | mmol/l |
| Process temperature | | 55° C. | |
| Current density | | 110 | mA/cm2 |
| Time | | 20 | min. |
| Layer thickness | | 20 | μm |

The calcium hydroxide layer has comparable properties to the calcium hydroxide layer from Example 1, but is not deposited directly on the electrically conductive material of the base body, but on the calcium phosphate layer, which in turn is deposited directly on the electrically conductive layer of the base body.

1-15. (canceled)

16. An implant or implant component, comprising
(a) a base body of an implant or an implant component, comprising an electrically conductive material at least on a surface; and
(b) a calcium hydroxide layer comprising calcium hydroxide, the calcium hydroxide layer being deposited on the electrically conductive material of the base body;
wherein the calcium hydroxide layer comprises calcium phosphate in a weight percentage less than the weight percentage of calcium hydroxide, the weight percentage being based on the total weight of the calcium hydroxide layer.

17. The implant or implant component according to claim 16, wherein the calcium phosphate comprised in the calcium hydroxide layer comprises a calcium phosphate selected from the group consisting of calcium dihydrogen phosphate, calcium hydrogen phosphate, calcium phosphate, hydroxyapatite, brushite, and combinations thereof

18. The implant or implant component according to claim 17, wherein the calcium phosphate comprised in the calcium hydroxide layer comprises hydroxyapatite and/or brushite.

19. The implant or implant component according to claim 16, wherein the base body comprises a material selected from the group consisting of metals, semi-metals, carbons, plastics, and ceramics.

20. The implant or implant component according to claim 16, wherein the electrically conductive material of the base body
(i) is deposited on the base body or is in one piece with the base body; and/or
(ii) is selected from the group consisting of metals, semi-metals, carbons and plastics.

21. The implant or implant component according to claim 16, wherein a calcium phosphate layer comprising calcium phosphate is deposited on the electrically conductive material of the base body.

22. The implant or implant component according to claim 21, wherein the calcium phosphate layer comprises calcium phosphate in a weight percentage that is higher than a weight percentage of calcium hydroxide in said layer, wherein the weight percentage relates to the total weight of the calcium phosphate layer.

23. The implant or implant component according to claim 16, wherein the calcium hydroxide layer
(i) is deposited by electrochemical deposition;
(ii) is present as a layer having an equal layer thickness over the entire extent of the layer; and/or
(iii) has a layer thickness in the range from 1 to 50 μm.

24. The implant or implant component according to claim 16, wherein the proportion of calcium phosphate in the calcium hydroxide layer is in the range of >0% by weight to <40% by weight.

25. A method for manufacturing an implant or an implant component, comprising the steps of
(a) providing a base body of an implant or an implant component, the base body comprising an electrically conductive material at least on a surface; and
(b) depositing a calcium hydroxide layer comprising calcium hydroxide to the electrically conductive material of the base body;
wherein the calcium hydroxide layer is deposited such that it comprises calcium phosphate in a weight percentage which is less than the weight percentage of calcium hydroxide, wherein the weight percentage is based on the total weight of the calcium hydroxide layer.

26. The method according to claim 25, wherein the calcium phosphate comprised in the calcium hydroxide layer comprises a calcium phosphate selected from the group consisting of calcium dihydrogen phosphate, calcium hydrogen phosphate, calcium phosphate, hydroxyapatite, brushite and combinations thereof.

27. The method according to claim 25, wherein a base body is provided, which comprises a material selected from the group consisting of metals, semi-metals, carbons, plastics, and ceramics.

28. The method according to claim 25, wherein a base body is provided, the electrically conductive material of which
(i) is deposited on the base body or is in one piece with the base body; and/or
(ii) is selected from the group consisting of metals, semi-metals, carbons and plastics.

29. The method according to claim 25, wherein a calcium phosphate layer comprising calcium phosphate is deposited on the electrically conductive material of the base body.

30. The method according to claim 29, wherein the calcium phosphate layer comprises calcium phosphate in a weight percentage that is higher than a weight percentage of calcium hydroxide in said layer, wherein the weight percentage relates to the total weight of the calcium phosphate layer.

31. The method of claim 29, wherein the calcium phosphate layer is
(i) deposited on the electrically conductive material of the base body by means of an electrochemical deposition or a plasma spraying process;
(ii) deposited as a layer having an equal layer thickness over the entire extent of the layer;
(iii) deposited as a porous layer; and/or
(iv) deposited up to a layer thickness in the range from 2 to 500 μm.

32. The method according to claim 25, wherein the calcium hydroxide layer is
(i) deposited by electrochemical deposition;
(ii) deposited as a layer having an equal layer thickness over the entire extent of the layer; and/or
(iii) deposited up to a layer thickness in the range from 1 to 50 μm.

33. The method according claim 25, wherein the proportion of calcium phosphate is in the range from >0% by weight to <10% by weight, based on the total weight of the calcium hydroxide layer.

34. The method according to claim 33, wherein the calcium phosphate is present in the form of particles in the calcium hydroxide layer.
